(19) United States
(12) Patent Application Publication
(10) Pub. No.: US 2021/0085596 A1
(43) Pub. Date: Mar. 25, 2021

(54) COMPOSITIONS FOR ORAL MUCOADHESIVE DOSAGE FORMS

(71) Applicant: Food Technology and Design, LLC, DBA FoodPharma, Santa Fe Springs, CA (US)

(72) Inventor: Timothy M. Marshall, Tucson, AZ (US)

(21) Appl. No.: 16/996,889

(22) Filed: Aug. 18, 2020

Related U.S. Application Data
(63) Continuation-in-part of application No. 16/673,125, filed on Nov. 4, 2019.
Application No. 16/673,125 is a continuation-in-part of application No. 16/673,038, filed on Nov. 4, 2019.
Application No. 16/673,038 is a continuation of application No. 16/128,349, filed on Sep. 11, 2018.
Application No. 16/128,349 is a continuation-in-part of application No. 16/673,083, filed on Nov. 4, 2019.
Application No. 16/673,083 is a continuation of application No. 16/128,349, filed on Sep. 11, 2018.
Application No. 16/128,349 is a continuation-in-part of application No. PCT/US2019/018414, filed on Feb. 18, 2019.
(60) Provisional application No. 62/985,585, filed on Mar. 5, 2020.
(60) Provisional application No. 62/971,023, filed on Feb. 6, 2020.
(60) Provisional application No. 62/965,440, filed on Jan. 24, 2020.
(60) Provisional application No. 62/933,762, filed on Nov. 11, 2019.
(60) Provisional application No. 62/910,093, filed on Oct. 3, 2019.
(60) Provisional application No. 62/910,090, filed on Oct. 3, 2019.
(60) Provisional application No. 62/910,081, filed on Oct. 3, 2019.
(60) Provisional application No. 62/696,955, filed on Jul. 12, 2018.
(60) Provisional application No. 62/696,955, filed on Jul. 12, 2018.
(60) Provisional application No. 62/632,043, filed on Feb. 19, 2018.

(51) Int. Cl. A61K 9/00; A61K 47/26; A61K 47/44; A61K 47/12; A61K 47/24; A61K 31/375; A61K 33/00; A61K 33/30; A61K 33/04; A61K 45/06; A61K 31/194; A61K 33/06; A61K 47/69; A61K 31/135; A61K 31/167; A61K 31/192; A61K 31/44; A61K 31/4402; A61K 31/4545; A61K 31/485; A61K 31/495; A61K 31/616; A61K 47/02; A61K 47/14; A61K 47/36

(52) U.S. Cl. CPC A61K 9/006; A61K 47/36; A61K 47/44; A61K 47/12; A61K 47/24; A61K 31/375; A61K 33/00; A61K 33/30; A61K 33/04; A61K 45/06; A61K 31/194; A61K 33/06; A61K 47/6903; A61K 9/0056; A61K 31/135; A61K 31/167; A61K 31/192; A61K 31/44; A61K 31/4402; A61K 31/4545; A61K 31/485; A61K 31/495; A61K 31/616; A61K 47/02; A61K 47/14; A61K 47/26

(57) ABSTRACT

The present disclosure relates to the field of oral mucoadhesive dosage forms and the type and amounts of structural components to improve mucoadhesion. The present disclosure further relates to combinations of active ingredients within a mucoadhesive dosage form.

### CROSS REFERENCE TO RELATED APPLICATIONS

The present invention claims priority to U.S. application Ser. Nos. 16/673,083 and 16/673,038, filed on Nov. 4, 2019, which are continuations of U.S. application Ser. No. 16/128,349, filed on Sep. 11, 2018, which claims priority to U.S. Provisional Application No. 62/696,955, filed on Jul. 12, 2018.

The present invention claims priority to U.S. Provisional Application No. 62/985,585 (filed on Mar. 5, 2020), U.S. Provisional Application No. 62/933,762 (filed on Nov. 11, 2019), U.S. Provisional Application No. 62/965,440 (Jan. 24, 2020), U.S. Provisional Application Nos. 62/910,093, 62/910,081, and 62/910,090 (filed on Oct. 3, 2019), U.S. Provisional Application No. 62/971,023 (filed on Feb. 6, 2020), U.S. application Ser. No. 16/673,125 (filed on Nov. 4, 2019), and International Application No. PCT/US2019/018414, filed on Feb. 18, 2019, which claims priority to U.S. Provisional Application No. 62/632,043, filed on Feb. 19, 2018.

### FIELD OF THE DISCLOSURE

The present invention relates to novel oral mucoadhesive dosage forms and compositions of active ingredients contained within.

### BACKGROUND OF THE DISCLOSURE

Mucoadhesion is a specific phenomenon of creating bonds during intimate contact between biological surfaces covered by a mucus layer and a mucoadhesive material. The oral bioavailability and uptake of small molecules (e.g. nutrients, nutraceuticals) is often limited by the short contact-time between the formulation and the oral mucosa, and a fast washout due to saliva flow.

Mucoadhesive dosage forms may be designed (e.g., gummy) to enable prolonged retention for increased small molecule absorption for improved therapeutic outcomes, efficacy, and consumer benefit. Application of dosage forms to mucosal surfaces may be of benefit to small molecules not amenable to the oral route, such as those that undergo acid degradation or extensive first-pass metabolism. Mucoadhesive-based formulations have shown enhanced bioavailability. Mucoadhesive small molecule delivery provides rapid absorption and improved bioavailability due to considerable surface area and high blood flow. Small molecule delivery across the mucosa bypasses gastrointestinal acid and enzymatic degradation, and first-pass hepatic metabolism (Shaikh R, et al. J Pharm BioalliedSci. 2011; 3(1):89-100).

Given the biodynamics of molecular transport being largely a function of a molecule's size (molecular weight; mol wt), charge, and lipophilicity, neutral, smaller molecules traverse both the paracellular and transcellular transport routes into the systemic circulation more readily than charged, larger molecules. This dramatic effect of molecular size/weight on bioavailability can be readily seen in the small molecule, vitamin B6 (pyridoxine) which has a molecular weight of 169 Da and a bioavailability of 75-100% from food or supplements. In contrast, and in alignment with the effect of molecular weight on bioavailability, the large-molecule, vitamin B12 (cobalamin) with a molecular weight of 1355 Da possesses an extremely low bioavailability of 1-2%. These two examples provide a general, quantitative template that can be applied to a wide variety of organic molecules of similar size/weight that demonstrates the inverse relationship of a molecule's size/weight on mucosal absorption (i.e. the ability of a small molecule to traverse the oral and GI mucosa by simple diffusion/paracellular transport) and bioavailability.

Deficiencies of existing mucosal delivery systems include short mucosal contact time and prolonged time required for absorption of ionized or larger molecular-weight compounds. What is needed is an improved mucosal delivery system with prolonged mucosal contact time with more rapid mucosal absorption of the delivered composition than currently available systems.

### SUMMARY OF THE DISCLOSURE

The present invention relates to oral mucoadhesive dosage forms and active ingredient compositions that may be within oral mucoadhesive dosage forms.

In an embodiment, the present invention provides for an oral mucoadhesive dosage form comprising a saccharide base comprising a saccharide, a surfactant, a lubricant, and an active ingredient composition, wherein the oral mucoadhesive dosage form adheres to the oral cavity.

In an embodiment, the present invention provides for an oral mucoadhesive dosage form comprising a plant-derived saccharide.

In an embodiment, the present invention provides for an oral mucoadhesive dosage form comprising a plant-derived surfactant.

In an embodiment, the present invention provides for an oral mucoadhesive dosage form comprising a plant-derived lubricant.

In an embodiment, the present invention provides for an oral mucoadhesive dosage form wherein the dosage form has a mass of about 4.5-5.8 grams.

In an embodiment, the present invention provides for an oral mucoadhesive dosage form further comprising an excipient.

In an embodiment, the present invention provides for an oral mucoadhesive dosage form further comprising a sweetener.

In an embodiment, the present invention provides for an oral mucoadhesive dosage form further comprising a colorant.

In an embodiment, the present invention provides for an oral mucoadhesive dosage form wherein the active ingredient composition comprises a composition of cannabinoid and a mineral chosen from the group consisting of magnesium, lithium, and zinc.

In an embodiment, the present invention provides for an oral mucoadhesive dosage form wherein the mineral comprises an organic chelated form of magnesium, lithium, or zinc.

In an embodiment, the present invention provides for an oral mucoadhesive dosage form further comprising a composition of vitamins and minerals.

In an embodiment, the present invention provides for an oral mucoadhesive dosage form wherein the composition of vitamins and minerals comprises at least one of about 50-3,000% the recommended daily dosage of each of vitamin B1, Vitamin B2, vitamin B3, vitamin B5, vitamin B6, and folic acid, about 0.1-100 mg PABA, about 0.5-10 mg boron, about 25-1000 mcg chromium, about 55-400 mcg selenium, about 100-2000 mg taurine, and about 25-250 mcg molybdenum.

In an embodiment, the present invention provides for an oral mucoadhesive dosage form wherein the composition of vitamins and minerals further comprises at least one of about 50-1000 mcg vitamin B12, about 25-200 mg coenzyme q10, about 25-500 mg alpha-lipoic acid, about 25-500 mg acetyl-L-carnitine, and about 50-500 mcg iodine.

In an embodiment, the present invention provides for an oral mucoadhesive dosage form, wherein the active ingredient composition of a mineral and a cannabinoid consists of a cannabinoid chosen from the group consisting of cannabigerol (CBG), cannabidiol (CBD), cannabinol (CBN), and beta-caryophyllene.

In an embodiment, the present invention provides for an oral mucoadhesive dosage form, wherein the active ingredient composition comprises a pharmaceutical.

In an embodiment, the present invention provides or an oral mucoadhesive dosage form wherein the saccharide comprises 50-70% of the saccharide base, the surfactant comprises 0.5-1.5% of the saccharide base, and the lubricant comprises 5-10% of the saccharide base.

In an embodiment, the present invention provides or an oral mucoadhesive dosage form wherein the saccharide comprises 55-65% of the saccharide base, the surfactant comprises 0.5-1.5% of the saccharide base, and the lubricant comprises 7.5-10% of the saccharide base.

In an embodiment, the present invention provides for an oral mucoadhesive dosage form comprising tapioca syrup, sunflower lecithin, palm oil, and an active ingredient combination, wherein the oral mucoadhesive dosage form adheres to the oral cavity.

In an embodiment, the present invention provides for an oral mucoadhesive dosage form, wherein the tapioca syrup comprises 55-65% of the saccharide base, the sunflower lecithin comprises 0.5-1.5% of the saccharide base, and the palm oil comprises 7.5-10% of the saccharide base.

### BRIEF DESCRIPTION OF THE FIGURES

FIG. 1 is a diagram illustrating trans-mucosal absorption of a composition from the oral cavity.

FIG. 2 is a diagram illustrating a mucoadhesive delivery system used in a buccal and gingival position within the oral cavity.

FIG. 3 is a diagram illustrating a mucoadhesive delivery system used in a sublingual position within the oral cavity.

FIG. 4. is a diagram illustrating a mucoadhesive delivery system used in a soft palatal position within the oral cavity.

### DETAILED DESCRIPTION

### Definitions

The term “oral” has its normal meaning in the art and is used herein to refer to the mouth. The term “oral cavity” is used herein to refer to the space within the mouth extending from the lips to the hard palate, along with the several types of tissues within the oral cavity. There are several types of mucous membranes within the interior of the oral cavity. Lining mucosa cover the parts of the mouth comprising the cheeks, floor of the mouth, and the lips. Masticatory mucosa is keratinized and lines the hard palate, the dorsum of the tongue, and gingiva. Specialized mucosa contain nerve endings for sensory perception and reside in the areas of the tongue with taste buds.

The term “mucoadhesive” is used herein to mean a property of an object or surface that adheres to a mucous membrane.

The term “adheres” is used herein to mean one surface or object sticking or clinging to a second surface or object. The mechanics of adhesion may be accomplished by any means.

The term “gummy” is used herein to mean a solid soft dosage form that is usually eaten or administered orally. Gummies may or may not be chewy or sticky inside the mouth.

The term “organic” has its normal meaning in the art and is used herein to mean derived from plants or animals.

The term “active ingredient” has its normal meaning in the art and is used herein to mean any ingredient that has a biological effect.

The term “pharmaceutical” has its normal meaning in the art and is used herein to mean a small molecule drug.

The term “cannabinoid” has its normal meaning in the art and is used herein to mean a group of chemicals found in the cannabis plant and can include, but is not limited to, hemp oil extract, cannabigerol, cannabinol, cannabidiol, and beta-caryophyllene.

The term “vitamin” has its normal meaning in the art and Is used herein to mean essential micronutrients. It should be noted that while this application may refer specifically to certain types of vitamins, the present inventor considers all forms of that vitamin to be within the disclosure of the present invention. As an illustrative example, inclusion of vitamin B12 in the present invention may include any of vitamin b12's four forms: methylcobalamin, adenosylcobalamin, hydroxycobalamin, and cyanocobalamin.

The term “excipient” has its normal meaning in the art and is used herein to mean any ingredient in a dosage form that is not an active ingredient.

The term “about” is used herein to mean within 10% of the stated amount.

Unless otherwise defined, all other scientific and technical terms have the same meaning as commonly understood to one of ordinary skill in the art. Such scientific and technical terms are explained in the literature, for example: Martin, 1990, Remington's Pharmaceutical Sciences, 18th Edition, Mack Publishing Co.

Oral Mucoadhesive Dosage Forms

The embodiments herein represent an oral mucoadhesive dosage form. In one embodiment, an oral mucoadhesive dosage form is provided using a saccharide base and an active ingredient composition described below. Persons having skill in the art will be aware of ingredients that may be used to form a purely saccharide-based oral mucoadhesive dosage form.

In another embodiment, an oral mucoadhesive dosage form is provided with an extended mucoadhesive residence time, maximizing oral mucosal, small molecule absorption. Key components may include the addition of a surfactant that boosts the mucoadhesive properties of the saccharide-base leading to a longer residence time, with a lubricant that increases the spreadable properties of the oral mucoadhesive dosage form and increases the surface area of interaction, maximizing oral mucosal absorption. Mass range per dosage form is about 4.5-5.8 grams, with the average mass per dosage form being about 5.5 grams. This allows for exceptional loading, with typical small molecule loading being in the range of about 2.0-2.4 grams per dosage form.

In another embodiment, an oral mucoadhesive dosage form is provided with shorter mucoadhesive residence time. Key components may include added disintegrants or texturizing agents to shorten residence time. Mass range per dosage form is about 2.0-2.5 grams, with average mass per dosage form being about 2.25 grams. This results in about 2.4 times less carrying capacity than certain alternative embodiments, and typical small molecule loading is in the range of about 0.8-0.9 grams per dosage form.

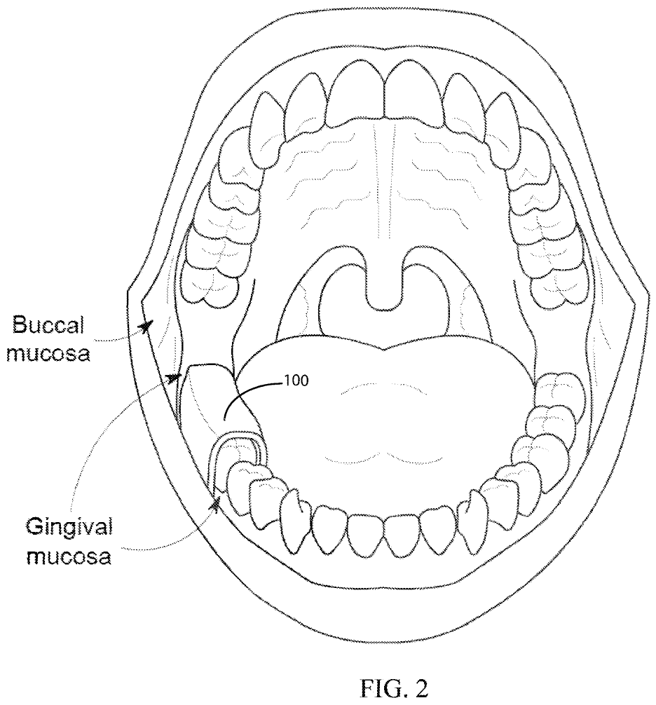

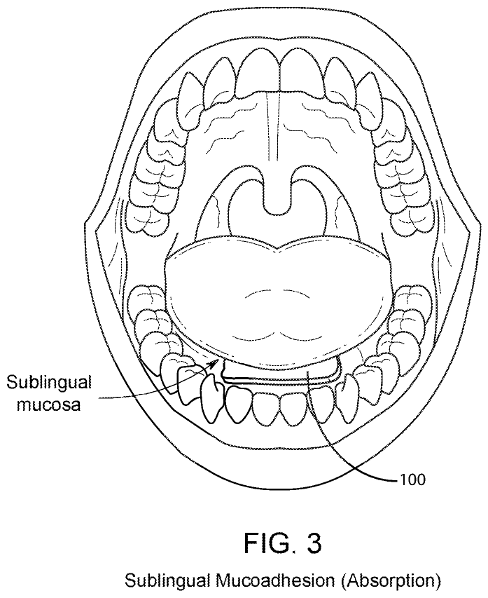

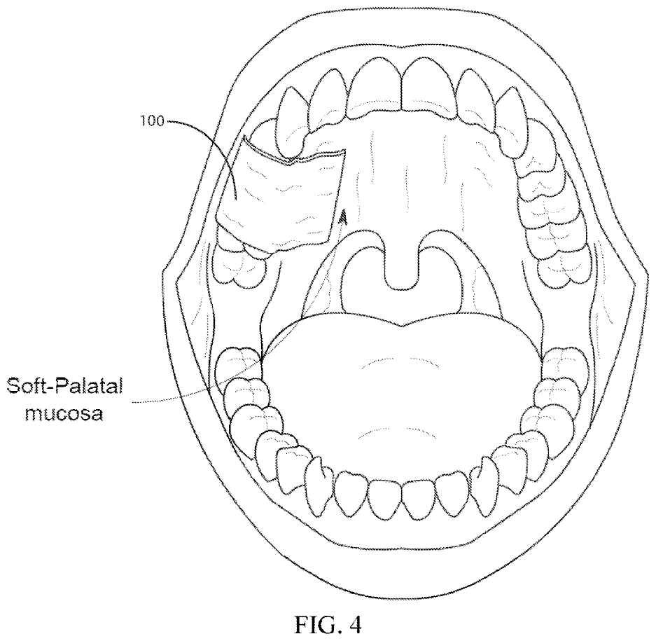

Oral mucoadhesive dosage forms may be configured to adhere to one or more different mucous membranes within the oral cavity. FIG. 2 illustrates an oral mucoadhesive dosage form configured to adhere to the buccal and gingival mucosa. FIG. 3, illustrates an oral mucoadhesive dosage form configured to adhere to the sublingual mucosa. FIG. 4 illustrates an oral mucoadhesive dosage form configured to adhere to the soft-palatal mucosa.

The oral mucoadhesive dosage form may comprise a saccharide base. The saccharide base may comprise any one or more of a combination of the following saccharide complexes or isolates: tapioca syrup; isomalto-oligosaccharide (IMO) syrup; powdered isomalto-oligosaccharide (IMO); honey; powdered honey; yacon syrup; agave syrup; corn syrup; glucose syrup; coconut sugar syrup; coconut sugar; date syrup; molasses; rice syrup; sugar cane syrup; raw cane sugar; cane sugar syrup; turbinado syrup; allulose syrup; maltitol syrup; polyglycitol syrup; sugar beet syrup; inulin syrup; powdered inulin; fibrosol; maltodextrin; dextrin; gum arabic; dextrose anhydrous; dextrose monohydrate; dried glucose syrup; sorghum syrup; tagatose syrup; and the following sugar alcohols: erythritol syrup; mannitol syrup; sorbitol syrup; or xylitol syrup; ethylene glycol; glycerol; erythritol; threitol; arabitol; xylitol; ribitol; mannitol; sorbitol; galactitol; fucitol; iditol; and inositol.

The saccharide used in the saccharide base may be an organic saccharide. Alternatively, in some embodiments the saccharide may be non-organic or synthetically derived. The amount of saccharide in the saccharide base may comprise 50-70% by weight or volume of the saccharide base. In another embodiment, the saccharide base comprises 55-65% by weight or volume of the saccharide base.

The saccharide base may also comprise a surfactant. Addition of a surfactant may increase the adhesive property of the dosage form, allowing for longer contact with the mucous membranes in the mouth. In some embodiments, the surfactant may be organically or non-organically derived. For example, lecithin may be used as an organic surfactant. Lecithin suitable for the present invention may be derived from soy, sunflower, cottonseed, rapeseed, eggs, canola, animal fat, or milk. The amount of surfactant in the saccharide base may comprise about 0.5-1.5% by weight or volume of the saccharide base.

The saccharide base may also comprise a lubricant. As mentioned above, addition of a lubricant increases the ability of the dosage form to spread, increasing the amount of surface area the dosage form is in contact with the mucous membranes of the mouth. In some embodiments, the lubricant may be organically or non-organically derived. For example, plant-derived oils may be used as a lubricant. Exemplary plant-derived oils suitable for use in the present invention include, but are not limited to: palm oil; coconut oil; sunflower oil; soy oil; canola oil; grapeseed oil; olive oil; flaxseed oil; avocado seed oil; and sesame seed oil. The amount of lubricant in the saccharide base may comprise about 5-10% by weight or volume of the saccharide base. In another embodiment, the amount of lubricant in the saccharide base may comprise about 7.5-10% by weight or volume of the saccharide base.

The listed exemplary saccharides, surfactants, and lubricants may be combined in any combination or one or more of them consistent with the present invention.

The active ingredients described below are formulated with one or more pharmaceutically acceptable excipients. An “excipient,” as used herein, has its normal meaning in the art and is any ingredient of an oral dosage form that is not an active ingredient (drug) itself. Excipients include, for example, binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere substantially with the biological activity of the active ingredients and that is not toxic to the subject, i.e., is a type of excipient and/or is for use in an amount which is not toxic to the subject. In the case of a pregnant human female subject, the pharmaceutically acceptable excipient is also not toxic to the embryo or fetus, i.e., a pharmaceutical excipient suitable for administration to a pregnant female. Thus, in dosage forms for administration to pregnant subjects, pharmaceutically acceptable excipients that have teratogenic properties and/or that are contraindicated for use in pregnancy should not be included.

Excipients are known in the art, and the present system is not limited in these respects. See, for example, Remington's Pharmaceutical Sciences, 18th Edition, A. Gennaro, Ed., Mack Pub. Co. (Easton, Pa., 1990), Chapters 88-91. In certain embodiments, one or more formulations of the dosage form include excipients, including for example and without limitation: one or more binders (binding agents); thickening agents; surfactants; diluents; release-delaying agents; colorants; fillers; disintegrants/dissolution promoting agents; lubricants; plasticizers; silica flow conditioners; glidants; anti-caking agents; anti-tacking agents; stabilizing agents; anti-static agents; swelling agents; and any combinations of one or more thereof. As those of skill would recognize, a single excipient can fulfill more than two functions at once, e.g., can act as both a binding agent and a thickening agent. Persons skilled in the art will also recognize that these terms are not mutually exclusive.

Useful diluents, e.g., fillers, may include, for example and without limitation: dicalcium phosphate; calcium diphosphate; calcium carbonate; calcium sulfate; lactose; cellulose; kaolin; sodium chloride; starches; powdered sugar; colloidal silicon dioxide; titanium oxide; alumina; talc; colloidal silica; microcrystalline cellulose; silicified micro crystalline cellulose; and combinations thereof. Fillers that can add bulk to tablets with minimal drug dosage to produce tablets of adequate size and weight include; croscarmellose sodium NF/EP (e.g., Ac-Di-Sol); anhydrous lactose NF/EP (e.g., Pharmatose™ DCL 21); and/or povidone USP/EP. In an embodiment, the diluent or filler is microcrystalline cellulose.

Binder materials employable in such formulations may include, for example and without limitation: starches (including corn starch and pregelatinized starch); gelatin; sugars (including sucrose, glucose, dextrose and lactose); polyethylene glycol; povidone; waxes; and natural and synthetic gums, e.g., acacia sodium alginate; polyvinylpyrrolidone; cellulosic polymers (e.g., hydroxypropyl cellulose, hydroxypropyl methylcellulose, methyl cellulose, hydroxyethyl cellulose, carboxymethylcellulose, colloidal silicon dioxide NF/EP (e.g., Cab-O-Sil™ M5P)); Silicified Microcrystalline Cellulose (SMCC) (e.g., Silicified microcrystalline cellulose NF/EP (e.g., Prosolv™ SMCC 90); and silicon dioxide; mixtures thereof; and the like); veegum; and combinations thereof.

Lubricants may include, for example: canola oil; glyceryl palmitostearate; hydrogenated vegetable oil (type I); magnesium oxide; magnesium stearate; mineral oil; poloxamer; polyethylene glycol; sodium lauryl sulfate; sodium stearate fumarate; stearic acid; talc and zinc stearate; glyceryl behapate; magnesium lauryl sulfate; boric acid; sodium benzoate; sodium acetate; sodium benzoate/sodium acetate (in combination); DL leucine; calcium stearate; sodium stearyl fumarate; mixtures thereof; and the like. In an embodiment, the lubricant is magnesium stearate.

Bulking agents may include, for example: microcrystalline cellulose, for example, AVICEL® (FMC Corp.) or EMCOCEL® (Mendell Inc.), which also has binder properties; dicalcium phosphate, for example, EMCOMPRESS® (Mendell Inc.); calcium sulfate, for example, COMPACTROL® (Mendell Inc.); and starches, for example, Starch 1500; and polyethylene glycols (CARBOWAX®).

Suitable disintegrating or dissolution promoting agents may include, but are not limited to: starches; clays; celluloses; alginates; gums; crosslinked polymers; colloidal silicon dioxide; osmogens; mixtures thereof; and the like, such as crosslinked sodium carboxymethyl cellulose (AC-DI-SOL®)), sodium croscarmelose, sodium starch glycolate (EXPLOTAB®, PRIMO JEL®) crosslinked polyvinylpolypyrrolidone (PLASONE-XL®), sodium chloride, sucrose, lactose and mannitol. In an embodiment, the disintegrating agent is sodium croscarmelose.

Antiadherents and glidants may include: talc; starches (e.g., cornstarch); celluloses; silicon dioxide; sodium lauryl sulfate; colloidal silica dioxide; and metallic stearates, among others.

Examples of silica flow conditioners include: colloidal silicon dioxide; magnesium aluminum silicate; and guar gum. In an embodiment, the silica flow conditioner is silicon dioxide.

Examples of stabilizing agents include: acacia; albumin; polyvinyl alcohol; alginic acid; bentonite; dicalcium phosphate; carboxymethylcellulose; hydroxypropylcellulose; colloidal silicon dioxide; cyclodextrins; glyceryl monostearate; hydroxypropyl methylcellulose; magnesium trisilicate; magnesium aluminum silicate; propylene glycol; propylene glycol alginate; sodium alginate; carnauba wax; xanthan gum; starch; stearate(s); stearic acid; stearic monoglyceride; and stearyl alcohol. In an embodiment, the stabilizing agent is magnesium trisilicate.

Optionally, a thickening agent can be added to provide the dosage form (e.g., gummy) with an accurately timed disintegration behavior. The dosage form optionally disintegrates at a rate which is sufficiently slow to permit it to be swallowed easily, but fast enough to give an excellent suspension in water within 60 seconds. The thickening agent may be, for example, talc USP/EP; a natural gum, such as guar gum or gum Arabic; or a cellulose derivative such as microcrystalline cellulose NF/EP (e.g., Avicel™ PH 102); methylcellulose; ethylcellulose; or hydroxyethylcellulose. A useful thickening agent is hydroxypropyl methylcellulose, an adjuvant which is available in various viscosity grades.

Suitable plasticizers include: acetylated monoglycerides; these can be used as food additives; Alkyl citrates, used in food packagings, medical products, cosmetics and children toys; Triethyl citrate (TEC); Acetyl triethyl citrate (ATEC), higher boiling point and lower volatility than TEC; Tributyl citrate (TBC); Acetyl tributyl citrate (ATBC), compatible with PVC and vinyl chloride copolymers; Trioctyl citrate (TOC), also used for gums and controlled release medicines; Acetyl trioctyl citrate (ATOC), also used for printing ink; Trihexyl citrate (THC), compatible with PVC, also used for controlled release medicines; Acetyl trihexyl citrate (ATHC), compatible with PVC; Butyryl trihexyl citrate (BTHC, trihexyl o-butyryl citrate), compatible with PVC; Trimethyl citrate (TMC), compatible with PVC; alkyl sulphonic acid phenyl ester, polyethylene glycol (PEG) or any combination thereof. Optionally, the plasticizer can comprise triethyl citrate NF/EP.

In some embodiments, the oral mucoadhesive dosage form may include a sweetener or colorant. The sweetener or colorant may be organic or non-organic. Suitable sweeteners include, but are not limited to: evaporated cane juice crystals; organic stevia (leaf) extract; monk fruit extract; artificial sweeteners (saccharin, acesulfame, aspartame, neotame, and sucralose); and natural and/or artificial flavors.

In one embodiment, the oral mucoadhesive dosage form comprises gelatin at about 0.4% by weight (range 0.2-1%), tapioca syrup at about 60.33% (range 48.3-72.4%), evaporated cane juice crystals at about 23.77% (range 19.0-28.51), mono and diglycerides at about 2.29% (range 1.83-2.75%), palm oil/shortening at about 7.31% (range 5.85-8.77%), lecithin at about 0.27% (range 0.216-0.324%), sea salt at about 0.55% (range 0.44-0.66%), citric acid at about 0.70% (range 0.66-0.84%), natural or artificial flavor at about 1.20% (0.96-1.44%), and natural or artificial color at about 0.80% (0.64-0.96%).

In another embodiment, the oral mucoadhesive dosage form comprises agar gum at about 7% by weight (range 2-15%), tapioca syrup at about 59.66% (range 47.7-71.6%), evaporated cane juice crystals at about 21.4% (range 17.1-25.7), palm oil/shortening at about 7.78% (range 6.22-9.34%), lecithin at about 1.1% (range 0.88-1.32%), sea salt at about 0.65% (range 0.52-0.78%), citric acid at about 1.18% (range 0.94-1.42%), natural or artificial flavor at about 1.18% (range 0.94-1.42%), and natural or artificial color at about 0.79% (range 0.63-0.95%).

In an embodiment, the oral mucoadhesive dosage form comprises agar gum and xanthan gum at about 3% each by weight (50/50 blend) (range 2-10% each), corn syrup at about 60% (range 48-72%), evaporated cane juice crystals at about 20% (range 16-24%), mono and diglycerides at about 2.29% (range 1.83-2.75%), palm oil/shortening at about 7.31% (range 5.85-8.77%), lecithin at about 0.27% (range 0.216-0.324%), sea salt at about 0.55% (range 0.44-0.66%), citric acid at about 0.65% (range 0.52-0.78%), natural or artificial flavor at about 0.65% (range 0.52-0.78%), and natural or artificial color at about 1.3% (range 1.04-1.56%).

In an embodiment, the oral mucoadhesive dosage form comprises pre-blended agar and xanthan gum at about 3% by weight (range 2-10%), tapioca syrup at about 43.4% (34.7-52.1%), cane juice crystals at about 43.4% (34.7-52.1%), mono and diglycerides at about 1.52% (range 1.22-1.82%), lecithin at about 0.20% (range 0.16-0.24%), and fruit juice concentrate at about 0.33% (range 0.066-0.396%), citric acid at about 0.65% (range 0.52-0.78%), natural or artificial flavor at about 0.65% (range 0.52-0.78%), and natural or artificial color at about 1.3% (range 1.04-1.56%).

In an embodiment, the oral mucoadhesive dosage form comprises pectin at about 5% by weight (range 1-10%), tapioca syrup at about 60.33% (range 48.3-72.4%), evaporated cane juice crystals at about 23.77% (range 19.0-28.51), mono and diglycerides at about 2.29% (range 1.83-2.75%), palm oil/shortening at about 7.31% (range 5.85-8.77%), lecithin at about 0.27% (range 0.216-0.324%), sea salt at about 0.55% (range 0.44-0.66%), citric acid at about 0.70% (range 0.66-0.84%), natural or artificial flavor at about 1.20% (0.96-1.44%), and natural or artificial color at about 0.80% (0.64-0.96%).

In an embodiment, the oral mucoadhesive dosage form comprises pectin at about 5% by weight (range 1-10%), tapioca syrup at about 43.4% (34.7-52.1%), evaporated cane juice crystals at about 43.4% (34.7-52.1%), mono and diglycerides at about 1.52% (range 1.22-1.82%), lecithin at about 0.20% (range 0.16-0.24%), and fruit juice concentrate at about 0.33% (range 0.066-0.396%), citric acid at about 0.65% (range 0.52-0.78%), natural or artificial flavor at about 0.65% (range 0.52-0.78%), and natural or artificial color at about 1.3% (range 1.04-1.56%).

In an embodiment, the oral mucoadhesive dosage form comprises tapioca starch at about 7.5% by weight (range 2-15%), tapioca syrup at about 60.33% (range 48.3-72.4%), evaporated cane juice crystals at about 23.77% (range 19.0-28.51), mono and diglycerides at about 2.29% (range 1.83-2.75%), palm oil/shortening at about 7.31% (range 5.85-8.77%), lecithin at about 0.27% (range 0.216-0.324%), sea salt at about 0.55% (range 0.44-0.66%), citric acid at about 0.70% (range 0.66-0.84%), natural or artificial flavor at about 1.20% (0.96-1.44%), and natural or artificial color at about 0.80% (0.64-0.96%).

In an embodiment, the oral mucoadhesive dosage form comprises tapioca starch at about 7.5% by weight (range 2-15%), tapioca syrup at about 43.4% (34.7-52.1%), evaporated cane juice crystals at about 43.4% (34.7-52.1%), mono and diglycerides at about 1.52% (range 1.22-1.82%), lecithin at about 0.20% (range 0.16-0.24%), and fruit juice concentrate at about 0.33% (range 0.066-0.396%), citric acid at about 0.65% (range 0.52-0.78%), natural or artificial flavor at about 0.65% (range 0.52-0.78%), and natural or artificial color at about 1.3% (range 1.04-1.56%).

In an embodiment, the oral mucoadhesive dosage form comprises about 40-70% by weight tapioca syrup, at about 10-40% cane or beet sugar, 1-3% mono and diglycerides, 2-12% palm oil, 0.1-1% sunflower lecithin, 0.1-0.8% salt, 0.1-1%) citric acid, 0.2-2% natural or artificial flavors, and 0.2-2% natural or artificial colors.

In an embodiment, the oral mucoadhesive dosage form comprises about 40-70% by weight corn syrup, at about 10-40% cane or beet sugar, 1-3% mono and diglycerides, 2-12% palm oil, 0.1-1% sunflower lecithin, 0.1-0.8% salt, 0.1-1% citric acid, 0.2-2% natural or artificial flavors, and 0.2-2% natural or artificial colors.

Active Ingredient Compositions

Magnesium, lithium, zinc, and cannabinoids (e.g. anandamide) exert a portion of their neuroprotective, antioxidant, anti-inflammatory effects through inhibition of the N-methyl-D-aspartate receptor (“NMDA”), with which magnesium (and/or lithium, zinc) interact as a receptor ligand, on post-synaptic cortical neurons of the central nervous system. With their innate antioxidant activity, and CB1/CB2 receptor binding affinity, cannabinoids are believed to exert their NMDA inhibitory effect through a different mechanism. NMDA receptors are ubiquitous throughout the brain and play a role in regulation of the excitatory state of post-synaptic neurons. NMDA receptors act as a cationic membrane “pore,” primarily for calcium ions although other cations such as sodium, zinc, and protons may pass into the cell. In conditions wherein the post-synaptic neuron is polarized and glutamate is absent from the synapse, a local negative membrane charge permits the pore to be blocked with a magnesium ion. Under conditions wherein (1) glutamate is present within the synapse at a sufficient concentration; and (2) the post-synaptic neuron is partially depolarized creating a neutral or relative positive membrane charge, the magnesium ion is displaced, the pore opens, and calcium ions are allowed to pass freely through the NMDA receptor into the cell. Once intracellular, calcium exerts a myriad of secondary effects, largely through its role as a secondary messenger and enzyme cofactor. Increased intracellular calcium leads to increased cellular enzyme activity of proteases, nucleases, and phospholipases, breaking down structural components and functional machinery of the cell and often leading to cell death.

Maintaining the baseline state of the NMDA receptor pore in a closed configuration, therefore, is important for the proper function and survival of a post-synaptic neuron. There are at least two potential sites of action to keep the receptor pore closed to influx of calcium and other cations into the neuron: (1) adequate-to-high synaptic magnesium concentrations; and (2) tyrosine-mediated phosphorylation of the NR2B receptor subunit.

Given a polarized or neutral post-synaptic cell membrane, in combination with adequate extracellular magnesium concentrations, magnesium may bind to the receptor pore and block the influx of calcium.

Several physiologic mechanisms resist a partially depolarized state in the post-synaptic cell membrane, keeping the pore closed to the influx of calcium an enhancing appropriate NMDA receptor function. One of these potentiating mechanisms is tyrosine-mediated phosphorylation of the NMDA receptor subunits, tending to “close” the receptor pore by causing an amphoteric shift in one or more protein subunits. Tyrosine phosphatase-mediated NR2B subunit phosphorylation potentiated by the lithium cation has been shown to cause depression of NMDA receptor currents.

For more rapid onset, and maximum therapeutic benefit and efficacy, lipophilic, uncharged, poorly-ionized, mineral chelates such as those bound to orotate or glycinate are the preferred vehicles (carriers) for efficient, mineral delivery and absorption (i.e. high-bioavailability). Small molecule, mineral chelates such as those bound to orotate (e.g. Li-orotate, mol wt: 162 Da; divalent ions, di-orotates of Ca, Mg, Zn<410 Da) or glycinate (e.g. bisglycinates of Ca, Mg, Cu, Mn, Mo, Se<250 Da) are absorbed via hydrophilic, paracellular transport and lipophilic, transcellular transport, and/or through transcellular, carrier-mediated orotate or glycine transporters.

A 1978 study comparing the bioavailability of lithium orotate to lithium carbonate demonstrated three times greater bioavailability for poorly-ionized (lipophilic) lithium orotate as compared to highly-ionized (hydrophilic) lithium carbonate. Kling M A, Manowitz P, Pollack I W, Rat brain and serum lithium concentrations after acute injections of lithium carbonate and orotate, J Pharm Pharmacol. 1978; 30(6):368-370. This study illustrates a primary distinction between the orotate vs carbonate forms of lithium, showing that the poorly-ionized, uncharged, orotate form more readily crosses lipophilic barriers (cell membranes) compared to the highly-ionized, charged, carbonate form, as evidenced by a three-fold higher level in the brain. With our present understanding of small-molecule bioavailability, this effect can be applied to a wide range of molecules with similar (low mol wt, neutral, uncharged, lipophilic; high-bioavailability) and dissimilar (high mol wt, ionized, charged, hydrophilic; low-bioavailability) physicochemical properties.

In further support of the effect of ionizability (i.e. ability to form charged ions) and molecular weight/size on bioavailability, a 2008 study investigating the acute uptake (i.e. bioavailability) of four different forms of zinc (e.g. oxide, picolinate, gluconate, and glycinate) found that zinc glycinate had the highest bioavailability. Gandia P, Bour D, Maurette J M, et al., A bioavailability study comparing two oral formulations containing zinc (Zn bis-glycinate vs. Zn gluconate) after a single administration to twelve healthy female volunteers, Int J Vitam Nutr Res. 2007; 77(4):243-248. The following is the ionizability and molecular weight for each form: Zn-oxide (highly-ionized; mol wt: 81 Da), Zn-picolinate (moderately ionized; mol wt: 310 Da), Zn-gluconate (highly-ionized; mol wt: 456 Da), and Zn-glycinate (poorly-ionized; mol wt: 214 Da). Plasma zinc rankings based on area under the curve, as well as by rank results per person, were: glycinate>gluconate>picolinate=oxide. A 43.4% increase in bioavailability was seen for zinc glycinate over the second, most-bioavailable form, zinc gluconate.

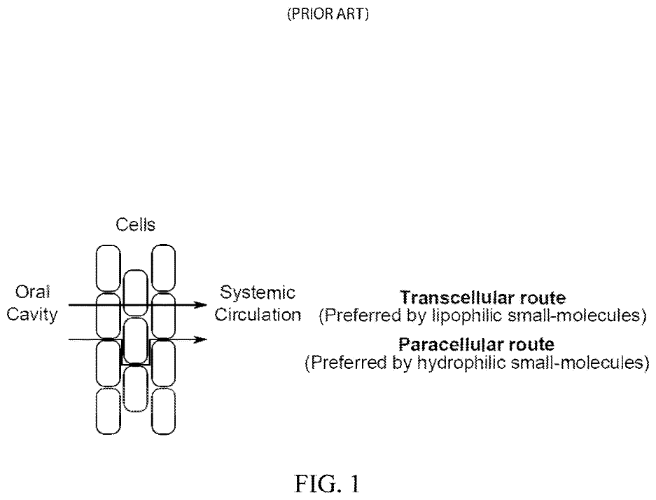

Neutral, small molecule, nutrient/nutraceutical transport occurs through both a paracellular transport mechanism across the tight junctions between cells, and directly across the lipid-bilayer of cell membranes via transcellular transport. Their low-molecular weight (i.e. small size) favors and promotes paracellular transport, while their neutral charge (coupled with small size) permits highly-efficient, transcellular (transmucosal) transport into the systemic circulation. FIG. 1 illustrates the transcellular route preferred by lipophilic molecules and the paracellular route preferred by hydrophilic molecules.

Stable, lipophilic, mineral chelates (e.g. glycinates or orotates) of magnesium, zinc, and lithium share similar physicochemical properties, in terms of size and lipophilicity, as that of known, highly-bioavailable, small molecules (mol wt<500 Da) such as caffeine (mol wt: 194 Da) and nicotine (mol wt: 162 Da), with molecular weights between 162 Da (lithium orotate) to 214 Da (zinc glycinate). Nutraceuticals such as terpenes (e.g. limonene, 136 Da), phenolics (e.g. cyanidin, 287 Da), and cannabinoids (e.g. anandamide, 348 Da) may also share similar physicochemical properties.

As discussed above, the invention relates to preparations of hemp oil, with or without a full-spectrum of naturally-occurring cannabinoids obtained from the hemp plant, in combination with magnesium and a blend of nutrients for oral delivery and absorption.

In an embodiment, the invention relates to preparations of hemp oil with a full-spectrum of naturally-occurring phytochemicals (e.g. terpenes, phenolics, cannabinoids) obtained from the hemp plant in combination with highly-bioavailable, nominally-ionized, lipophilic magnesium (e.g., malate, laminate, or glycinate) and a blend of vitamins, minerals, nutrients, or nutraceuticals for oral (e.g. gingival, buccal) delivery and absorption as a central nervous system neurotrophic and neuroprotectant and topical administration for reducing local pain and inflammation, and methods of use.

In another embodiment, the preparation of is limited to one or more specific cannabinoids that may or may not be derived from hemp oil. These cannabinoids include, but are not limited to, cannabigerol (CBG), cannabidiol (CBD), cannabinol (CBN), and beta-caryophyllene.

A wide range of primary central neurological diseases and secondary conditions manifest cognitive, memory, motor, and sensory impairment as primary and debilitating symptoms. A few non-limiting examples of such diseases and conditions include: amyotrophic lateral sclerosis (“ALD”); Parkinson's Disease (“PD”); Alzheimer's Disease (“AD”); post-traumatic stress disorder (“PTSD”); attention deficit hyperactivity disorder (“ADHD”); depression/anxiety; and tinnitus. Examples of secondary conditions include: traumatic brain injury (“TBI”); chronic post-traumatic or post-surgical neuropathic pain; acute or chronic exposure to certain toxins, for example mercury and ethanol; and cerebral ischemia.

Cannabinoids and magnesium exert a myriad of antioxidant, anti-inflammatory benefits on the brain and nervous system, skin, and possess wide-ranging, general health benefits.

Magnesium exerts a portion of its neuroprotective, antioxidant, anti-inflammatory effects through inhibition of the N-methyl-D-aspartate receptor (“NMDA”), with which magnesium (and/or lithium, zinc) interact as a receptor ligand, on post-synaptic cortical neurons of the central nervous system. NMDA receptors are ubiquitous throughout the brain and play a role in regulation of the excitatory state of post-synaptic neurons. NMDA receptors act as a cationic membrane “pore,” primarily for calcium ions although other cations such as sodium, zinc, and protons may pass into the cell. In conditions wherein the post-synaptic neuron is polarized and glutamate is absent from the synapse, a local negative membrane charge permits the pore to be blocked with a magnesium ion. Under conditions wherein (1) glutamate is present within the synapse at a sufficient concentration; and (2) the post-synaptic neuron is partially depolarized creating a neutral or relative positive membrane charge, the magnesium ion is displaced, the pore opens, and calcium ions are allowed to pass freely through the NMDA receptor into the cell. Once intracellular, calcium exerts a myriad of secondary effects, largely through its role as a secondary messenger and enzyme cofactor. Increased intracellular calcium leads to increased cellular enzyme activity of proteases, nucleases, and phospholipases, breaking down structural components and functional machinery of the cell and often leading to cell death.

Maintaining the baseline state of the NMDA receptor pore in a closed configuration, therefore, may be important for the proper function and survival of a post-synaptic neuron. There are at least two potential sites of action to keep the receptor pore closed to influx of calcium and other cations into the neuron: (1) adequate-to-high synaptic magnesium concentrations; and (2) tyrosine-mediated phosphorylation of the NR2B receptor subunit.

Given a polarized or neutral post-synaptic cell membrane, in combination with adequate extracellular magnesium concentrations, magnesium binds to the receptor pore and blocks influx of calcium.

Several physiologic mechanisms resist a partially depolarized state in the post-synaptic cell membrane, keeping the pore closed to the influx of calcium an enhancing appropriate NMDA receptor function. One of these potentiating mechanisms is tyrosine-mediated phosphorylation of the NMDA receptor subunits, tending to “close” the receptor pore by causing an amphoteric shift in one or more protein subunits. Tyrosine phosphatase-mediated NR2B subunit phosphorylation potentiated by the lithium cation has been shown to cause depression of NMDA receptor currents.

The present invention seeks to militate against activation of a final common pathway for neuronal cell injury and death—elevated intracellular calcium levels—by impeding permeability of the NMDA receptor to calcium.

Magnesium concentrations in a neuronal synapse are necessary to saturate the post-synaptic population of NMDA receptors, therein keeping the receptor pores closed to the influx of extracellular calcium ions when the post-synaptic neuron is in a partially polarized state. Hypomagnesaemia is associated with a plethora of symptomatic neurological abnormalities, such as depression, anxiety, sleep disturbances, hyperreflexia, tremor, confusion, hallucinations, convulsions, hyperacusis, nystagmus, tetany, delirium tremens, and extrapyramidal disorders.

Trans-mucosal absorption of elemental magnesium is greatly increased by combining elemental magnesium with a chelate. In some embodiments, magnesium chelating compound is a salt of orotic acid. In some embodiments, magnesium chelating compound is a salt of succinic acid, aspartic acid, threonic acid, gluconic acid, lysinic acid, malic acid, tauric acid, or citric acid. It is anticipated that as experimentation and research in the art of enhanced trans-mucosal magnesium absorption progresses, other chelating compounds may be used as a chelating compound, in some embodiments.

The total concentration of elemental magnesium per dose of the neuroprotective preparation is calculated to provide adequate intracerebral levels of magnesium without being so high as to increase the risk of toxicity from hypermagnesaemia. Accordingly, the amount of elemental magnesium, in some embodiments, is between approximately 10 milligrams and 400 milligrams.

The synergistic nutrients and/or nutraceuticals used with this novel, mucoadhesive delivery system can by comprised, but not limited to, any one or combination of the following: vitamins, minerals, nutrients, or nutraceuticals used for their therapeutic, neurotrophic and/or neuroprotective benefits, as previously described above. Such vitamins, minerals, nutrients, or nutraceuticals include, but are not limited to: magnesium; kelp; vitamin A; vitamin B1; vitamin B2; vitamin B3; vitamin B5; vitamin B6; vitamin B12; folic acid; vitamin C; vitamin D2; vitamin D3; vitamin E; vitamin K2; boron; copper; zinc; manganese; selenium; molybdenum; chromium; iodine; biotin; Para-aminobenzoic acid (PABA); coenzyme Q10; alpha lipoic acid; acetyl-L-carnitine; wild blueberry extract; french melon extract (e.g. SOD); kelp; terpenes; phenolics; and cannabinoids.

In some embodiments, the composition of vitamins, minerals, nutrients, or nutraceuticals comprise (per serving): magnesium glycinate, malate, taurinate, or threonate, 25-400 mg; lithium orotate, 0.5-20 mg; boron glycinate, 0.5-10 mg; copper glycinate, 0.25-2 mg; zinc glycinate, 5-20 mg; manganese glycinate, 0.5-5 mg; selenium glycinate, 25-200 mcg; molybdenum glycinate, 25-200 mcg; and chromium polynicotinate, 25-400 mcg; kelp, 10-100 mg; vitamin B1, 1-25 mg; vitamin B2, 1-25 mg; vitamin B6, 1-25 mg; Vitamin B3, 5-100 mg; vitamin B5, 5-200 mg; Vitamin B12 as methylcobalamin, hydroxocobalamin, adenosylcobalamin; folate as folic acid, methyl-folate, or folinic acid, 25-400 mcg; 25-1,000 mcg; non-GMO vitamin C as ascorbic acid, ascorbyl palmitate, or ascorbates, 50-1,000 mg; vitamin D3, 500-5,000 IU; and nutraceuticals such as terpenes (e.g. limonene), phenolics (e.g. cyanidin), and cannabinoids (e.g. anandamide).

In some embodiments, the composition of vitamins, minerals, nutrients, or nutraceuticals comprises: magnesium as glycinate, malate, taurinate or threonate (as well as other organic or inorganic forms of the mineral), 10-400 mg; kelp, 5-100 mg; vitamin A as retinyl palmitate or acetate or beta-carotene, 500-5,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 5-5,000 mcg as cyanocobalamin or methylcobalamin or hydroxocobalamin or adenosylcobalamin; folic acid or methyl-folate or folinic acid, 10-1,000 mcg; 25-1,000 mcg; non-GMO vitamin C, 25-1,000 mg; vitamin D3, 500-10,000 IU; vitamin E as d-alpha tocopherol or dl-alpha tocopherol and/or mixed tocopherols and/or tocotrienols, 10-400 iu for alpha-tocopherol and 5-500 mg for mixed tocopherols including gamma-tocopherol or the family of tocotrienols; boron as glycinate (as well as other organic or inorganic forms of the mineral), 0.5-10 mg; copper as bisglycinate (as well as other organic or inorganic forms of the mineral), 0.1-5 mg; zinc as bisglycinate (as well as other organic or inorganic forms of the mineral), 2.5-50 mg; manganese as bisglycinate (as well as other organic or inorganic forms of the mineral), 0.5-10 mg; selenium as glycinate (as well as other organic or inorganic forms of the mineral), 10-400 mcg; molybdenum as glycinate (as well as other organic or inorganic forms of the mineral), 10-400 meg; and chromium as nicotinate glycinate (as well as other organic or inorganic forms of the mineral), 10-1,000 mcg; and nutraceuticals such as terpenes (e.g. limonene), phenolics (e.g. cyanidin), and cannabinoids.

In some embodiments, the composition of vitamins and minerals comprises at least one of about 50-3,000% the recommended daily dosage of vitamin B1, Vitamin B2, vitamin B3, vitamin B5, vitamin B6, and folic acid; about 0.1-100 mg PABA; about 0.5-10 mg boron; about 25-1,000 mcg chromium; about 55-400 mcg selenium; about 100-2,000 mg taurine; about 25-250 mcg molybdenum; about 50-1,000 mcg vitamin B12; about 25-200 mg coenzyme Q10; about 25-500 mg alpha-lipoic acid; about 25-500 mg acetyl-L-carnitine; and about 50-500 mcg iodine.

### EXAMPLES

The following Examples are intended as illustrative and to assist in describing the invention and are meant for illustrative purposes only and are not meant to limit the invention in any way.

HempMag Mood™ Composition

| | Ingredient | Weight (m/m %) | % |
|---|---|---|---|
| Saccharide | | | |
| Base | | | |
| | TapiOK Tapioca Syrup | 504.0 | 63.72 |
| | 40DE Ciranda | | |
| | Evaporated Cane Juice | 184.6 | 23.34 |
| | Crystals | | |
| | Palm AP Shortening- | 69.3 | 8.76 |
| | #736 (Colombus) | | |
| | MP: 114-123F | | |
| | Sunflower Lecithin | 9.5 | 1.20 |
| | Water | 16.0 | 2.02 |
| | Sea Salt | 7.6 | 0.96 |
| | Total | 791.0 | 100 |
| Total | | | |
| Gummy | | | |
| | Saccharide Base | 643.0 | 80.27 |
| | Citric Acid Anhydrous | 13.8 | 1.72 |
| | (Batory-Sucroal) | | |
| | Vitamin Premix | 17.8 | 2.22 |
| | WE-30387 (Wright) | | |
| | DiMagnesium Malate | 22.0 | 2.75 |
| | (20% Mg) (Albion) | | |
| | Magnesium Taurinate | 53.5 | 6.68 |
| | (7.5% Mg) (Nutrisol | | |
| | Solutions) | | |
| | VO Hemp Oil (19% CBD) | 2.10 | 0.26 |
| | (Verdant Oasis) | | |
| | Lot: VOHO-AV18K12-1M | | |
| | 92.144GMOF-Natural | 10.4 | 1.30 |
| | Mango WONF (Flavorchem) | | |
| | Nat. Dark Sweet Cherry | 13.8 | 1.72 |
| | Flavor WONF S09-337-1 | | |
| | (Signature Flavors) | | |
| | Natural Peach Flavor | 9.1 | 1.14 |
| | WONF O.C. 68.728 | | |
| | (Flavorchem) | | |
| | Beet Juice Concentrate | 9.1 | 1.14 |
| | (BEETCONC) (Flavorchem) | | |
| | ButterBuds Non Dairy | 6.4 | 0.80 |
| | Cream Concentrate | | |
| | (Masking) 49500 | | |
| | Total | 801.0 | 100.00 |

HempMag FOCUS™ Composition

| | Ingredients | Weight (m/m %) | % |
|---|---|---|---|
| Saccharide | | | |
| Base | | | |
| | TapiOK Tapioca Syrup | 639.6 | 63.77 |
| | 40DE Ciranda | | |
| | Evaporated Cane Juice | 234 | 23.36 |
| | Crystals | | |
| | Palm AP Shortening- | 88 | 8.77 |
| | #736 (Colombus) | | |
| | MP: 114-123F | | |
| | Sunflower Lecithin | 12 | 1.20 |
| | Water | 20.3 | 2.02 |
| | Sea Salt | 8.8 | 0.88 |
| | Total | 1003 | 100 |
| Total | | | |
| Gummy | | | |
| | Saccharide Base | 656.0 | 81.98 |
| | Citric Acid Anhydrous | 13.6 | 1.70 |
| | (Batory-Sucroal) | | |
| | Vitamin Premix | 17.8 | 2.22 |
| | WE-30387 (Wright) | | |
| | DiMagnesium Malate | 22.0 | 2.75 |
| | (20% Mg) (Albion) | | |
| | Magnesium Taurinate | 53.4 | 6.67 |
| | (7.5% Mg) (Novel - | | |
| | Nutrisol Solutions) | | |
| | VO Hemp Oil (19% CBD) | 2.10 | 0.26 |
| | (Verdant Oasis) | | |
| | Lot: VOHO-AV18K12-1M | | |
| | Boron Glycine (10% Boron) | 0.74 | 0.09 |
| | WS-81041 (Wright) | | |
| | Beet Juice Concentrate | 9.2 | 1.15 |
| | (BEETCONC) (Flavorchem) | | |
| | Natural Big Stick Type | 22.2 | 2.77 |
| | Flavor CI-BS-20897 | | |
| | (Custom Flavors) | | |
| | ButterBuds Non Dairy | 3.2 | 0.40 |
| | Cream Concentrate | | |
| | (Masking) 49500 | | |
| | Total | 800.2 | 100.00 |

HempMag Calm™ Composition

| | Ingredient | Weight (m/m %) | % |
|---|---|---|---|
| Saccharide | | | |
| Base | | | |
| | TapiOK Tapioca Syrup | 455.0 | 60.95 |
| | 40DE Ciranda | | |
| | Evaporated Cane Juice | 199.0 | 26.66 |
| | Crystals | | |
| | Palm AP Shortening- | 62.6 | 8.39 |
| | #736 (Colombus) | | |
| | MP: 114-123F | | |
| | Sunflower Lecithin | 8.5 | 1.14 |
| | Water | 14.4 | 1.93 |
| | Sea Salt | 7.0 | 0.94 |
| | Total | 746.5 | 100 |
| Total | | | |
| Gummy | | | |
| | Saccharide Base | 606.0 | 75.69 |
| | Citric Acid | 13.6 | 1.70 |
| | (Batory - Sucroal) | | |
| | Vitamin Premix | 17.8 | 2.22 |
| | WE-30387 (Wright) | | |
| | DiMagnesium Malate | 22.0 | 2.75 |
| | (20% Mg) (Albion) | | |
| | Magnesium Taurinate | 53.5 | 6.68 |
| | (7.5% Mg) (Novel - | | |
| | Nutrisol Solutions) | | |
| | VO Hemp Oil (19% CBD) | 2.10 | 0.26 |
| | (Verdant Oasis) | | |
| | Lot: VOHO-AV18K12-1M | | |
| | GNT Exberry Shade | 14.4 | 1.80 |
| | Red 153330 | | |
| | Ascorbic Acid (99%) | 4.20 | 0.52 |
| | WS-7396 or WS-9014 | | |
| | or WS-10000 or WS-9758 | | |
| | Ascorbyl Palmitate | 29.8 | 3.72 |
| | WS-81137 (95%) | | |
| | Citrus Bio Complex | 5.0 | 0.62 |
| | “50” HP - 50% | | |
| | Citrus Bioflavonoids | | |
| | (Using 45%) (21843) | | |
| | (Stauber - Brewster | | |
| | Foods) | | |
| | Natural Grape Flavor | 12.0 | 1.50 |
| | CI - GR-13239 | | |
| | (Custom Flavors) | | |
| | Nat. Apple Flavor 10-00300 | 7.8 | 0.97 |
| | or Organic Nat. Apple Flavor | | |
| | S07-106-13 (Signature Flavors) | | |
| | Nat. Cranberry Flavor | 6.0 | 0.75 |
| | 26.130 (Flavorchem) | | |
| | ButterBuds Non Dairy | 6.4 | 0.80 |
| | Cream Concentrate | | |
| | (Masking) 49500 | | |
| | Total | 800.6 | 100.00 |

HempMag CoolDoWn™ Composition

| | Ingredient | Weight (m/m %) | % |
|---|---|---|---|
| Saccharide | | | |
| Base | | | |
| | TapiOK Tapioca Syrup | 467.0 | 58.68 |
| | 40DE Ciranda | | |
| | Evaporated Cane Juice | 171.1 | 21.50 |
| | Crystals | | |
| | Palm AP Shortening- | 64.3 | 8.08 |
| | #736 (Colombus) | | |
| | MP: 114-123F | | |
| | Sunflower Lecithin | 8.8 | 1.11 |
| | Water | 14.8 | 1.86 |
| | Sea Salt | 5.8 | 0.73 |
| | Item# 0018B50G | 64.0 | 8.04 |
| | Dark Cocoa - Guittard | | |
| | Total | | |
| | | 796 | 100.00 |
| Total | | | |
| Gummy | | | |
| | Saccharide Base | 648.0 | 80.93 |
| | Vitamin Premix | 17.8 | 2.22 |
| | WE-30387 (Wright) | | |
| | DiMagnesium Malate | 22.0 | 2.75 |
| | (NLT 20% Mg) (Albion) | | |
| | Magnesium Taurinate 8% | 53.5 | 6.68 |
| | (NLT 7.5% Mg) (Novel - | | |
| | Nutrisol Solutions) | | |
| | VO Hemp Oil (Using 19% | 2.10 | 0.26 |
| | CBD, actual 20%) | | |
| | (Verdant Oasis) | | |
| | Lot: VOHO-AV18K12-1M | | |
| | Vitamin Premix (non-gmo) | 4.2 | 0.52 |
| | WE-30442 (Wright) | | |
| | Citrus Bio Complex | 5.0 | 0.62 |
| | “50” HP - 50% | | |
| | Citrus Bioflavonoids | | |
| | (Using 45%) (21843) | | |
| | (Stauber - Brewster | | |
| | Foods) (Active) | | |
| | Ascorbyl Palmitate | 16.6 | 2.07 |
| | WS-81137 (95%) | | |
| | Nat. Chocolate Flavor | 18.6 | 2.32 |
| | 10-00209 (S09-36-1) | | |
| | (Signature) | | |
| | Natural Vanilla Fl. | 6.5 | 0.81 |
| | WONF GMOF 93.8081 | | |
| | GM (Flavorchem) | | |
| | ButterBuds Non Dairy | 6.4 | 0.80 |
| | Cream Concentrate | | |
| | (Masking) 49500 | | |
| | Total | 800.7 | 100.00 |

HempMag Immunity™ Composition

| | Ingredient | Weight (m/m %) | % |
|---|---|---|---|
| Saccharide | | | |
| Base | | | |
| | TapiOK Tapioca Syrup | 457.0 | 60.94 |
| | 40DE Ciranda | | |
| | Evaporated Cane Juice | 199.9 | 26.66 |
| | Crystals | | |
| | Palm AP Shortening- | 62.9 | 8.39 |
| | #736 (Colombus) | | |
| | MP: 114-123F | | |
| | Sunflower Lecithin | 8.6 | 1.15 |
| | Water | 14.5 | 1.93 |
| | Sea Salt | 7.0 | 0.93 |
| | Total | 750 | 100.00 |
| Total | | | |
| Gummy | | | |
| | Saccharide Base | 614.0 | 76.63 |
| | Citric Acid | 8.6 | 1.07 |
| | (Batory - Sucroal) | | |
| | Vitamin Premix | 17.9 | 2.23 |
| | WE-30387 (Wright) | | |
| | DiMagnesium Malate | 22.0 | 2.75 |
| | (20% Mg) (Albion) | | |
| | Magnesium Taurinate | 53.5 | 6.68 |
| | (7.5% Mg) (Novel - | | |
| | Nutrisol Solutions) | | |
| | VO Hemp Oil (19% CBD) | 2.10 | 0.26 |
| | (Verdant Oasis) | | |
| | Lot: VOHO-AV18K12-1M | | |
| | Vitamin Premix (non-gmo) | 4.2 | 0.52 |
| | WE-30442 (Wright) | | |
| | Citrus Bio Complex | 5.2 | 0.65 |
| | “50” HP - 50% | | |
| | Citrus Bioflavonoids | | |
| | (Using 45%) (21843) | | |
| | (Stauber - Brewster | | |
| | Foods) | | |
| | Ascorbyl Palmitate | 16.6 | 2.07 |
| | WS-81137 (95%) | | |
| | Vitamin A Palmitate | 1.9 | 0.24 |
| | WE-101191 Non-GMO | | |
| | (250,000 IU/g) (Wright) | | |
| | Vit D3 WE-101952 | 4.2 | 0.52 |
| | (100,000 IU/g) (Wright) | | |
| | Vitamin E Natural 700 | 10.3 | 1.29 |
| | Non-GMO SD F080674 | | |
| | (700 IU/g) (Watson) | | |
| | Zinc Citrate Dihydrate | 4.2 | 0.52 |
| | Purified WS-2541 | | |
| | (Wright) (31.3% Zinc) | | |
| | Nat Blueberry Flavor | 11.9 | 1.49 |
| | 22-12-0032 (Weber) | | |
| | Natural Grape Flavor | 6.3 | 0.79 |
| | CI-GR-13239 (Custom) | | |
| | Exberry Shade | 12.0 | 1.50 |
| | “Purple Plum” (GNT) | | |
| | ButterBuds Non Dairy | 6.4 | 0.80 |
| | Cream Concentrate | | |
| | (Masking) 49500 | | |
| | Total | 801.3 | 100.00 |

In a study conducted at a neurology clinic in Tucson, Ariz., five participants received a dose equivalent to 4 HempMag COOLDOWN™ gummies supplying 200 mg of magnesium (from malate, taurinate) and 5.0 mg of CBD (from 50 mg of organic European hemp oil extract). This trial assessed overall effectiveness in terms of a self-reported greater sense of well-being.

A scientifically validated General Well-Being Survey was given to participants before and after taking the “HempMag:” supplement. Study participants previously reported neck stiffness. After administration, study participants reported “having no muscle tension, I can feel at this moment”, feeling “better, calm, and clear-headed,” “I feel completely calm. No stress. Focus seems to be a lot better,” “no pain, left shoulder injury/inflammation is no longer there after taking supplement,” “overall sensation is good/great. I feel like I can take on the world,” “acted as a mildly sedative without tiredness or cognitive reduction,” “I noticed I was more focused and clear-headed,”“might be psychological, but I feel better,” and similar responses that indicated a calming, anti-inflammatory effect.

The embodiments and examples set forth herein were presented to enable those of ordinary skill in the art to make and use said embodiments. However, those of ordinary skill in the art will recognize that the foregoing description and examples have been presented for the purposes of illustration and example only. The description as set forth is not intended to be exhaustive or to limit the claims to the precise form disclosed. Many modifications and variations are possible in light of the teachings above.

1. An oral mucoadhesive dosage form comprising: a saccharide base comprising a saccharide, a surfactant, a lubricant, and an active ingredient composition, wherein the oral mucoadhesive dosage form adheres to the oral cavity.

2-15. (canceled)

16. The oral mucoadhesive dosage form of claim 1, wherein the saccharide comprises about 50-70% of the saccharide base, the surfactant comprises about 0.5-1.5% of the saccharide base, and the lubricant comprises about 5-10% of the saccharide base.

17. The oral mucoadhesive dosage form of claim 16, wherein the saccharide comprises about 55-65% of the saccharide base, the surfactant comprises about 0.5-1.5% of the saccharide base, and the lubricant comprises about 7.5-10% of the saccharide base.

18. An oral mucoadhesive dosage form comprising tapioca syrup, sunflower lecithin, palm oil, and an active ingredient combination, wherein the oral mucoadhesive dosage form adheres to the oral cavity.

19. The oral mucoadhesive dosage form of claim 18, wherein the tapioca syrup comprises about 55-65% of the saccharide base, the sunflower lecithin comprises about 0.5-1.5% of the saccharide base, and the palm oil comprises about 7.5-10% of the saccharide base.

20. An active ingredient composition comprising citric acid, about 10-400 mg magnesium maleate, about 10-400 mg magnesium taurinate, and 50 mg hemp oil.

21. The active ingredient composition of claim 20, further comprising about 0.5-20 mg boron glycinate.

22. The active ingredient composition of claim 20, further comprising about 50-1,000 mg vitamin C.

23. The active ingredient composition of claim 22, further comprising about 500-5,000 IU vitamin A, about 500-5,000 IU vitamin D3, about 5-500 mg vitamin E, and about 5-20 mg zinc.

24. The oral mucoadhesive dosage form of claim 1, wherein the active ingredient composition comprises citric acid, magnesium maleate, magnesium taurinate, and hemp oil.

25. The oral mucoadhesive dosage form of claim 24, wherein the active ingredient composition further comprises boron glycinate.

26. The oral mucoadhesive dosage form of claim 24, wherein the active ingredient composition further comprises vitamin C.

27. The oral mucoadhesive dosage form of claim 26, wherein the active ingredient composition further comprises vitamin A, vitamin D3, vitamin E, and zinc.
